(19) United States
(12) Patent Application Publication
(10) Pub. No.: US 2005/0080279 A1
(43) Pub. Date: Apr. 14, 2005

(54) Process for production of fatty acid esters

(76) Inventors: Giovanni Barbieri, Vic (AU); Giacomo Salvatore Gasparini, Vic (AU); Peter Ronald Wrigley, Vic (AU); Peter William Jones, Vic (AU); John David Morrison, Vic (AU); Robert William Killick, Vic (AU)

Correspondence Address: FOLEY AND LARDNER;SUITE 500, 3000 K STREET NW, WASHINGTON, DC, 20007, US

(21) Appl. No.: 10/498,789

(22) PCT Filed: Dec. 13, 2002
(86) PCT No.: PCT/AU02/01687

(30) Foreign Application Priority Data
Dec. 13, 2001 (FR) PR 9462

(51) Int. Cl. C07C 51/43

(52) U.S. Cl. 554/174

(57) ABSTRACT

A process for producing C1-C4 alkyl esters of fatty acids comprising the steps of: (a) reacting glycerine with a glyceride mixture containing levels of free fatty acids which inhibit trans-esterification with alkaline catalysts until the level of free fatty acids is reduced sufficiently to enable the use of an alkaline trans-esterification catalyst; (b) reacting the mixture resulting from step (a) with one or more alkaline trans-esterification catalysts and one or more C1-C4 alcohols until a mixture of one or more C1-C4 alkyl esters of fatty acids and crude glycerine forms; (c) recovering the one or more C1-C4 alkyl esters of fatty acids; and (d) recovering the crude glycerine, wherein at least a portion of the crude glycerine recovered in step (d) is used in step (a) of a subsequent process.

### FIELD OF THE INVENTION

The invention relates to a process for production of esters of fatty acids. More particularly, it relates to a cost-effective process for production of esters of fatty acids from glycerides which contain high levels of free fatty acids.

### BACKGROUND OF THE INVENTION

In this specification, where a document, act or item of knowledge is referred to or discussed, this reference or discussion is not an admission that the document, act or item of knowledge or any combination thereof was at the priority date:

(a) part of common general knowledge; or

(b) known to be relevant to an attempt to solve any problem with which this specification is concerned.

Esters of fatty acids are useful chemicals in many areas of industry and have generated much interest as alternative fuels in recent times. Such esters are often produced by the trans-esterification of triglycerides from animal or vegetable sources with C1-C4 alcohols to produce C1-C4 alkyl esters of fatty acids, along with crude glycerine released as a by-product. A variety of catalysts, including acidic catalysts, enzymes, metal salts or alkaline catalysts, are available for trans-esterification. The alkaline catalysts such as sodium or potassium hydroxides or alkoxides are preferred because they are efficient, easily separated from the product and compatible with conventional reactor systems.

It is known that triglyceride mixtures from animal or vegetable sources contain free fatty acids (FFA) as well as mono- and di-glycerides. Oils which are crude or old, such as gut tallow or used frying oils, often have higher levels of FFA. The term “glyceride mixture” is used in this description to refer to fatty materials containing mono-, di- and tri-glycerides as well as FFA.

If there are high levels of FFA in the glyceride mixture, that is levels greater than 4%, difficulties can result in the trans-esterification process since the FFA will react with alkaline catalysts to form soaps. These soaps not only cause deactivation of the catalyst but also cause additional problems during further processing and purifying of the C1-C4 alkyl esters of fatty acids, because they tend to limit the release of crude glycerine and generate emulsion phases during washing cycles. The level of FFA in the glyceride mixture should be below about 4% and preferably below about 2% by weight to enable alkaline trans-esterification to take place efficiently.

It is possible to lower the FFA content of a glyceride mixture by alkali refining prior to trans-esterification , whereby the FFAs are selectively removed from the glyceride mixture as water soluble soaps. However, whilst alkali refining is suitable for removal of small amounts of FFA, oils with higher levels of FFA are not suited to this process because of high yield losses and poor phase separations associated with soapy emulsions.

Another attempt to lower the FFA content is disclosed in UK patent no 612,667. The process disclosed involves the conversion of substantially all of the fatty acid components into lower alkyl esters using an acidic catalyst. The process described is typical of acid catalysed trans-esterification processes in that it (1) requires a very large excess of alcohol which is not economical, (2) is not compatible with steel based reactor systems and (3) results in ester products which contain high levels of undesired FFA which can be removed by alkali refining but this process is inefficient by commercial standards.

Other attempts to reduce FFA content have involved the use of enzymes, in particular lipase, however these attempts have not provided results which are commercially acceptable.

There is thus a need for an efficient process for reducing high levels of free fatty acids in glyceride mixtures to enable the glyceride mixtures to be reacted with C1 to C4 alcohols in the presence of alkaline catalysts to produce C1-C4 alkyl esters of fatty acids.

### SUMMARY OF THE INVENTION

It has been found that if a glyceride mixture containing levels of FFA which inhibit trans-esterification with alkaline catalysts is reacted with glycerine (which is available as a by-product from this process) prior to adding the C1-C4 alcohol and catalyst, the FFAs can be reduced to a level which enables the efficient use of alkaline trans-esterification catalysts.

According to the invention there is provided a process for producing C1-C4 alkyl esters of fatty acids comprising the steps of:

(a) reacting glycerine with a glyceride mixture containing levels of free fatty acids which inhibit trans-esterification by alkaline trans-esterification catalysts until the level of free fatty acids is reduced sufficiently to enable the use of an alkaline trans-esterification catalyst;

(b) reacting the mixture resulting from step (a) with one or more alkaline trans-esterification catalysts and one or more C1-C4 alcohols until a mixture of one or more C1-C4 alkyl esters of fatty acids and crude glycerine forms;

(c) recovering the one or more C1-C4 alkyl esters of fatty acids; and

(d) recovering the crude glycerine

wherein at least a portion of the crude glycerine recovered in step (d) is used in step (a) of a subsequent process.

The glyceride mixture may be any such mixture known to those skilled in the art, including glycerides of animal, vegetable or synthetic origin. The glyceride mixture may be selected from virgin fatty products such as crude or refined tallow or vegetable oils or from used materials such as yellow grease (used frying oil) or acid oils (by-product of soap, oil processing industries or from this process). The levels of free fatty acids which inhibit trans-esterification with alkaline catalysts are known to those skilled in the art. Typically, the glyceride mixture used in the process according to the invention contains levels of FFA greater than 4% by weight of the glyceride mixture. Preferably the levels of FFA are in the range of from 4 to 75% by weight. More preferably, the levels of FFA are in the range of from 4 to 30% by weight.

The word “glycerine” when used in the context of step (a) refers to any glycerine containing mixture including crude glycerine recovered from step (d) which consists of more than 50% by weight glycerine. The glycerine may also contain some soap, alcohol and other fatty material. Mineral salts and acids are undesirable in the glycerine as they may inhibit the reaction or adversely affect the processing equipment.

The amount of crude glycerine recovered in step (d) is likely to be significantly larger than the amount required in step (a) and thus only the amount required will be used. Any excess crude glycerine recovered from step (d) may be stored for later use, used in another process or else sold. In some instances, the amount of contaminants in the crude glycerine recovered from step (d) may make it unsuitable as the sole source of glycerine in step (a) and it will be necessary to use some glycerine from another source. For example, if the glyceride mixture had a very high level of FFA then it would not be appropriate to use solely crude glycerine recovered from step (d) which had high levels of contaminants.

The amount of glycerine used in step (a) will depend on the level of FFA in the glyceride mixture. Typically for each 1% by weight of FFA in the glyceride mixture, 0.1 to 0.5% by weight of available glycerine would be required to reduce the FFA to below 2% by weight. Preferably, the amount of available glycerine is about 0.2% by weight for each 1% by weight of FFA in the glyceride mixture. Once the glyceride mixture contains less than 2% by weight FFA then an alkaline trans-esterification catalyst may be used. If the glyceride mixture contains alkyl esters of fatty acids then such alkyl esters should be considered as FFA when calculating the required amount of glycerine. This is because the alkyl ester may react with the glycerine to form glyceride releasing the alcohol which may be distilled from the reaction mixture with the water vapour.

A person skilled in the art will know that the reaction in step (a) may be conducted under a variety of temperature and pressure conditions. For example a temperature above about 180° C. at reduced pressure is commonly used to aid the removal of water produced during the reaction. Another example of suitable conditions for the reaction in step (a) is a temperature of about 220° C. at ambient pressure. The use of an unreactive atmosphere (eg nitrogen) or agitation of the reaction mixture may be useful to assist in the removal of water from the reaction. Optionally, a solvent may be added to the mixture to form a constant boiling azeotrope to assist removal of water.

A person skilled in the art will know that the reaction in step (b) may be conducted under a variety of temperature and pressure conditions. For example a temperature in the range from 75 to 85° C. at ambient pressure is commonly applied for one hour with agitation. Increased pressure may enable the reaction to proceed more quickly or completely. The reaction may then be left to rest to allow the crude glycerine to separate out by gravity or alternatively the crude glycerine may be separated by centrifugal separation or by plate separation.

A person skilled in the art will know of procedures for recovering the C1-C4 alkyl esters of fatty acids and crude glycerine in steps (c) and (d). For example, the mixture from step (b) may be left for two hours without agitation during which time the mixture will separate into two layers. The denser layer will be the crude glycerine (which is suitable for use as glycerine in (a)) and the lighter layer the C1-C4 alkyl esters of fatty acids. Typically the amount of crude glycerine generated from this process is more than is required for re-use in step (a) of a subsequent process and the amount of C1-C4 alkyl esters of fatty acids recovered is similar to the quantity of the original glyceride mixture.

Once separated, these products may then be used directly in this or another process or else purified according to standard procedures for sale or another use. For example, the crude glycerine may be acidified, defatted and (partially or completely) dried. Steam distillation and/or vacuum distillation and/or carbon decolourisation may be used to improve the glycerine. The fatty ester may be further reacted, water washed, dried, deodorised, distilled or decolourised.

Suitable alkaline trans-esterification catalysts are known to those skilled in the art and include sodium or potassium alkoxides or hydroxides. Alkoxides are preferred due to speed and efficiency of reaction. Typically, the amount of alkaline catalyst required to effect trans-esterification is in the range from 0.2 to 2% by weight of the glyceride mixture.

C1-C4 alcohols which are suitable for use in the process of the invention will be known to those skilled in the art. For example, methanol or ethanol may be used. A minimum of three moles of C1-C4 alcohol to each mole of triglyceride (assuming that the glyceride mixture with now low FFA is predominantly triglyceride) is required to effect the trans-esterification. Typically excess alcohol is used in this process to ensure completeness of reaction. Excess alcohol may be recovered from the mixture for reuse. Alternatively, the alcohol and alkaline catalyst can be reacted with the glyceride mixture more than once with removal of crude glycerine between applications.

In a preferred embodiment, any fatty glycerides or FFA recovered from purification of the C1-C4 alkyl esters of fatty acids (eg extracted from water washings) or from the crude glycerine can be appropriately treated (acidified and washed) and used as part of the glyceride mixture of a subsequent process according to the invention.

### DESCRIPTION OF THE DRAWING

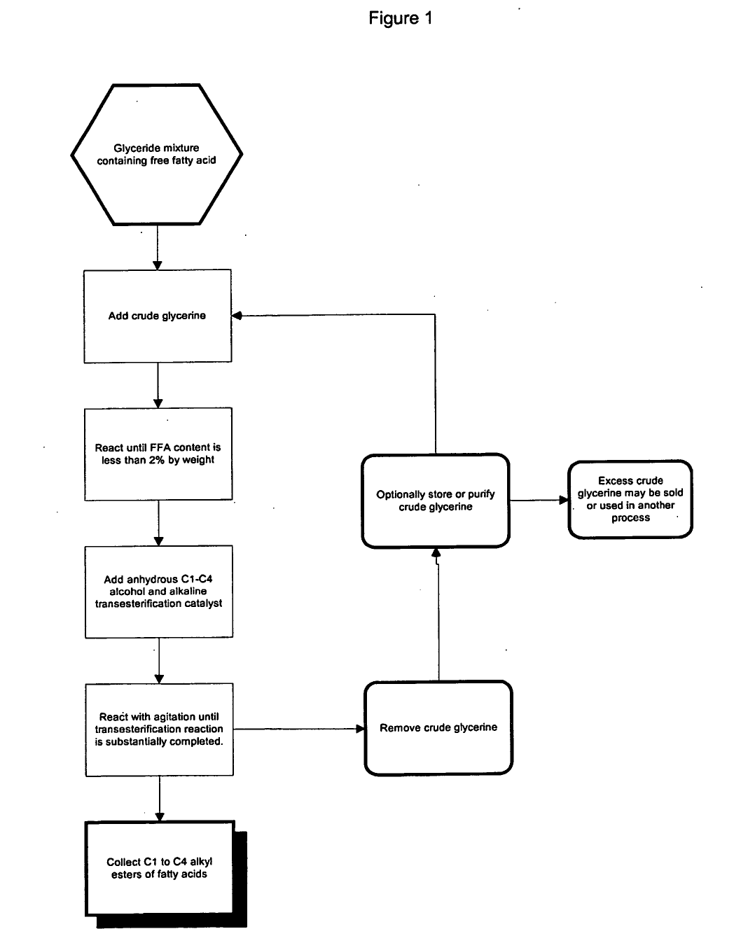

The invention will now be further described with reference to FIG. 1 which is a flowchart showing one embodiment of a process according to the invention.

### DETAILED DESCRIPTION OF THE DRAWING

In this embodiment, the process starts with a glyceride mixture containing levels of FFA which inhibit trans-esterification with alkaline catalysts, for example, about 20% by weight FFA. Examples of such glyceride mixtures are gut tallow or used frying oil.

The glyceride mixture was reacted with crude glycerine at a temperature above 180° C. and under reduced pressure until the level of FFA was below 2% by weight.

The mixture was then cooled to 75 to 85° C. and one or more anhydrous C1-C4 alcohols were added, for example ethanol, with an alkaline trans-esterification catalyst such as sodium methoxide. This mixture was then reacted at a temperature in the range of 75 to 85° C. for one hour with agitation. The reaction was then left to rest for two hours without agitation to allow the crude glycerine to separate as the denser phase which was then removed. This crude glycerine is a useful by-product, some of which was then used to react with further glyceride mixture containing high levels of FFA in a subsequent process. The rest may be stored, sold or used in another process.

The C1-C4 alkyl esters of fatty acids were then purified by removal of excess alcohol and catalyst, water washed and then dried.

While the process described here is indicative of a batch process, the separation and return of a portion of crude glycerine to react with the feedstock would be equally valid for a continuous or semi-continuous process.

### EXAMPLES

The invention will now be further explained and illustrated by reference to the following non-limiting examples.

### Example 1

| Components | Properties |
|---|---|
| Glyceride Mixture | Crude tallow mixture with 20% FFA and 5% |
| | unsaponifiables |
| Glycerine | Crude Glycerine derived from trans-esterification of |
| | ethyl canolate comprising 85% glycerine |
| Alcohol | Methylated Spirits 100SG:F3 ex CSR Distilleries |
| | Ethanol with 2% added methanol with maximum |
| | water content of 0.5%. |
| Catalyst | 30% Sodium Methylate in Methanol ex BASF |

Reduction of FFA

Glyceride mixture (180 kg) was reacted with the crude glycerine (100 kg) at 200-220° C. for 12 hours at ambient pressure with gentle nitrogen sparge in which time the level of FFA decreased from 20 to 4.3%. Additional crude glycerine (20 kg) was added and the reaction continued in the same temperature range for a further 8 hours to give a level of FFA of 2.7%. Again crude glycerine (20 kg) was added and a further 8 hours reaction resulted in a level of FFA of 1.8%.

Trans-Esterification with Alcohol

The resultant glyceride mixture with a level of FFA of 1.8% (approx 1920 kg) was reacted with methylated spirits (300 kg) and sodium methylate solution (58 kg) at 75-80° C. for 1 hour. The reaction mixture was allowed to stand for 2 hours and then crude glycerine (320 kg) was separated off by gravity.

The resultant mixture of ethyl and methyl esters of fatty acids was reacted with additional methylated spirits (80 kg) and sodium methylate solution (16 kg) at 75-80° C. for 1 hour in order to convert any remaining unreacted glycerides to fatty ester. The reaction mixture was then subjected to a series of water washes to remove soap, FFA, catalyst and other impurities then dried at 130° C. and ambient pressure to give a mixture of mixture of ethyl and methyl esters of fatty acids suitable for use as biodiesel. (Note that the washing process was not optimised. The efficiency of this process was compromised by the high level of unsaponifiables in the glyceride mixture.)

Results

| | | | DIN* | |
|---|---|---|---|---|
| | | | 51606 | |
| | | | Septem- | |
| | | | ber 1997 | |
| Test | Unit | Method | spec. | Results |
| Flash Point | ° C. | ASTM D93 | min 110 | 186 |
| Density | g/mL | ASTM D4052 | 0.875- | 0.8745 |
| | | | 0.90 | |
| Water Content | Ppm | ASTM E1064 | max 300 | 493 |
| Ash | % wt | ASTM D482 | 0.03 | 0.0250 |
| BS & W | % vol | ASTM D1796 | | <0.01 |
| Viscosity | cSt | ASTM D445 | 3.5-5.0 | 5.41 |
| @40° C. | | | | |
| CFPP | ° C. | IP 309 | summer 0 | +9 |
| | | | winter −20 | |
| Sulphur | % wt | ASTM D 4294 | 0.01 | <0.01 |
| Carbon Residue | % wt | ASTM D524 | | 0.047 |
| Particulate | mg/L | ASTM D5452* | 20 mg/kg | 16.5 |
| Contamination | | | | |
| Copper | degree of | ASTM D130 | 1 | 1a |
| Corrosion | corrosion | | | |
| Neutralisation | mgKOH/g | ASTM D974 | max 0.5 | 0.45 |
| Number | | | | |
| Iodine Number | — | AOCS Cd 1d-92 | max 115 | 43 |
| Phosphorus | mg/kg | ASTM D3231 | max 10 | <0.2 |
| Alkali Content | mg/kg | (1) | Max 5 | <2 |
| (Na + K) | | | | |

*Note that standards vary from country to country and are regularly being revised. The DIN standard relates typically to methyl esters of rapeseed oil and may not be applicable to alkyl esters derived from other feedstock.

### Example 2

| Components | Properties |
|---|---|
| Glyceride Mixture | Crude tallow mixture with 22% FFA and |
| | 0.85% unsaponifiables |
| Glycerine | Crude Glycerine derived from transesterification |
| | of ethyl canolate |
| | comprising 80.5% glycerine and 52 mg/g |
| | alkali calculated as KOH |
| Alcohol | Methylated Spirits 100SG:F3 ex CSR |
| | Distilleries |
| | Ethanol with 2% added methanol with |
| | maximum water content of 0.5%. |
| Catalyst | 30% Sodium Methylate in Methanol ex |
| | BASF |

Reaction Process
Reduction of FFA

Glyceride mixture (1000 g) was reacted with the crude glycerine (45 g) at 220-240° C. for 3 hours at ambient pressure with gentle nitrogen sparge in which time the level of FFA decreased from 22 to 4.0%. Reaction proceeded with vacuum applied for further 3 hours resulting in 1016 g of glyceride mixture with a level of FFA of 1.0%.

Trans-Esterification with Alcohol

The resultant glyceride mixture (1016 g with a level of FFA of 1.0%) was reacted with methylated spirits (167 g) and sodium methylate solution (28 g) at 75-80° C. for 1 hour. The reaction mixture was allowed to stand for 2 hours and then crude glycerine (235 g) was separated off by gravity.

The resultant mixture of ethyl and methyl esters of fatty acids was reacted with additional methylated spirits (40 g) and sodium methylate solution (9 g) at 75-80° C. for 1 hour in order to convert any remaining unreacted glycerides to fatty ester. The reaction mixture was then subjected to a series of water washes to remove soap, FFA, catalyst and other impurities then dried at 130° C. and ambient pressure to give a mixture of ethyl and methyl esters of fatty acids (774 g) suitable for use as biodiesel.

Results

| Test | Unit | Result |
|---|---|---|
| Acid Value | mgKOH/g | 0.2 |
| Moisture | % w/w | 0.02% |
| Clarity@20 C. | N/A | Clear and Bright |
| SG@20 C. | g/ml | 0.867 |
| Colour (Gardner) | N/A | 5 |
| FTIR | N/A | matches ethyl tallowate |

The word ‘comprising’ and forms of the word ‘comprising’ as used in this description does not limit the invention claimed to exclude any variants or additions.

Modifications and improvements to the invention will be readily apparent to those skilled in the art. Such modifications and improvements are intended to be within the scope of this invention.

1. A process for producing C1-C4 alkyl esters of fatty acids comprising the steps of:
(a) reacting glycerine with a glyceride mixture containing levels of free fatty acids which inhibit trans-esterification by alkaline trans-esterification catalysts until the level of free fatty acids is reduced sufficiently to enable the use of an alkaline trans-esterification catalyst;
(b) reacting the mixture resulting from step (a) with one or more alkaline trans-esterification catalysts and one or more C1-C4 alcohols until a mixture of one or more C1-C4 alkyl esters of fatty acids and crude glycerine forms;
(c) recovering the one or more C1-C4 alkyl esters of fatty acids; and
(d) recovering the crude glycerine
wherein at least a portion of the crude glycerine recovered in step (d) is used in step (a) of a subsequent process.

2. A process according to claim 1 wherein the glyceride mixture is selected from the group consisting of virgin fatty products such as crude or refined tallow or vegetable oils, from used materials such as yellow grease or acid oils, and mixtures thereof.

3. A process according to claim 1 wherein the glyceride mixture contains levels of free fatty acids equal to or greater than 4% by weight.

4. A process according to claim 3 wherein the glyceride mixture contains levels of free fatty acids in the range from 4 to 75% by weight.

5. A process according to claim 4 wherein the glyceride mixture contains levels of free fatty acids in the range from 4 to 30% by weight.

6. A process according to claim 1 wherein the level of free fatty acid in step (a) is reduced to less than 2% by weight.

7. A process according to claim 1 wherein the ratio of free fatty acid in the glyceride mixture to glycerine is in the range of from 1:0.1 to 1:0.5 by weight.

8. A process according to claim 7 wherein the ratio of free fatty acid in the glyceride mixture to glycerine is 1:0.2 by weight.

9. A process according to claim 1 wherein step (a) occurs at a temperature above about 180° C. and at a reduced pressure.

10. A process according to claim 1 wherein step (a) occurs at a temperature at about 220° C. and at ambient pressure.

11. A process according to claim 1 wherein agitation is used in step (a) to assist in removal of water from the reaction mixture.

12. A process according to claim 1 wherein an unreactive atmosphere is used in step (a).

13. A process according to claim 1 wherein step (b) occurs at a temperature in the range from 75 to 85° C., at ambient pressure for one hour with agitation.

14. A process according to claim 1 wherein steps (c) and (d) occur using gravity to separate the crude glycerine and C1-C4 alkyl esters of fatty acids into two layers which are then recovered.

15. A process according to claim 1 wherein steps (c) and (d) occur using centrifugal separation to separate the crude glycerine and C1-C4 alkyl esters of fatty acids which are then recovered.

16. A process according to claim 1 wherein steps (c) and (d) occur using plate separation to separate the crude glycerine and C1-C4 alkyl esters of fatty acids which are then recovered.

17. A process according to claim 1 wherein the C1-C4 alcohol is selected from the group consisting of methanol, ethanol and mixtures thereof.

18. A process according to claim 1 wherein the amount of C1-C4 alcohols is in excess of three moles of C1-C4 alcohol to each mole of glyceride.

19. A process according to claim 1 wherein the alkaline trans-esterification catalyst is an alkoxide or hydroxide.

20. A process according to claim 19 wherein the alkaline trans-esterification catalyst is an alkoxide.

21. A process according to any one of the preceding claims claim 1 wherein the amount of alkaline trans-esterification catalyst is in the range of 0.2 to 2% by weight of the glyceride mixture.

22. A process according to any one of the preceding claims claim 1 wherein the excess crude glycerine recovered in step (d) is further purified for sale or another use.
